(19) United States
(12) Patent Application Publication
(10) Pub. No.: US 2008/0020437 A1
(43) Pub. Date: Jan. 24, 2008

(54) APPARATUS AND METHOD FOR PRODUCING FUEL ETHANOL FROM BIOMASS

(76) Inventor: John J. SAVARESE, Collegeville, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/779,914

(22) Filed: Jul. 19, 2007

Related U.S. Application Data
(60) Provisional application No. 60/831,949, filed on Jul. 20, 2006.
(60) Provisional application No. 60/837,284, filed on Aug. 14, 2006.
(60) Provisional application No. 60/853,545, filed on Oct. 23, 2006.

(51) Int. Cl. C12P 7/10; C12M 1/02

(52) U.S. Cl. 435/165; 435/289.1

(57) ABSTRACT

Cellulosic biomass at the growing site is pulverized and hydrolyzed in collectors which transfer the comminuted biomass into pretreatment units. In the pretreatment units enzymes and/or other agents are added to the pretreatment units to disassociate the lignin from the cellulose and hemicellulose, and to further decompose the biomass to simple sugars. The sugar solution is transferred to a fermentation unit and fermenting microorganisms are added to produce ethanol. Preferably vegetable oil or other ethanol-miscible liquid is added to provide a medium to extract the ethanol from the solution for transfer to a device where heating or distillation removes the ethanol which is collected for use as a liquid fuel. All of the units are preferably mounted for easy deployment to various locations in the vicinity of the numerous growing sites which produce the biomass.

### CROSS REFERENCE TO RELATED APPLICATIONS

This application claims priority under 35 U.S.C. 119 (e) to U.S. Provisional Applications 60/831,949 filed Jul. 20, 2006, 60/837,284 filed Aug. 14, 2006 and 60/853,545 filed Oct. 23, 2006, the entire disclosures of which are incorporated by reference herein.

### FIELD OF THE INVENTION

The present invention relates to mobile apparatus for producing fuel ethanol from biomass and methods of using the same.

### BACKGROUND OF THE INVENTION

Processing of sugar cane or corn kernels to beverage ethanol is an ancient process which today is commercialized to produce ethanol for use as an alternative fuel. While Brazil, for example, uses sugar cane to produce fuel ethanol, corn is the feedstock prevalent in the United States. However, corn grain is a major component of animal feed and human food and cannot be relied on as the major feedstock for fuel ethanol production. It is recognized that cellulose (biomass) in unused crop residue or new crops grown simply as biomass is the feedstock of choice for the United States. Conversion of biomass to fuel ethanol presents challenges.

The first and most important challenge in processing biomass into fuel ethanol results from the nature of biomass itself. Biomass is the plant vegetation remaining after the crop product is removed and is known chemically as lignocellulose. For example, after corn ears are harvested, the remaining biomass of stalk, leaves, and husks (if left) is called corn stover which is composed of cellulose, hemicelluloses, and lignin, all three of which are included in the term lignocellulose. Cellulose itself is a polymer composed of monomers of the six carbon simple sugar glucose (a hexose) bound together by beta linkages. Starch is similar but has alpha linkages. Humans produce the enzyme to break the alpha bonds; however, we do not produce the ‘cellulase’ enzymes to hydrolyze beta linkages. Ruminants such as cows can do this with their complex stomach digestive systems which contain microbes which produce cellulases. Cellulase(s) are a mixture of enzymes which work in concert to breakdown cellulose and hemicellulose. Hemicellulose is like cellulose but is a polymer of primarily five carbon sugars (pentoses), mostly xylose. Lignin is a complex polymer not of sugars but of other chemicals which intertwine with cellulose and hemicelluloses in plant cell walls to provide structural strength and waterproofing. In order to ferment lignocellulose materials like corn stover, straw, grasses, etc., it is necessary to pretreat the biomass with chemicals which open the lignocellulose structure allowing the cellulose and hemicelluloses to be exposed to water for hydrolysis, i.e., breakage of the beta linkage bonds. Chemicals, enzymes, temperature, solvents, pressure, and other factors have been and continue to be examined to determine optimum conditions for hydrolysis of biomass to fermentable sugars. However, recent work has shown that treatment of biomass with low concentrations of lye (2-5% NaOH) under moderate temperature and pressure is very effective in conjunction with use of cellulases. This affords the potential of biomass pretreatment done directly on the farm to be followed by fermentation to produce ethanol.

Another major challenge is the harvesting, transportation, and mechanical comminution of biomass. The current model for the biomass to ethanol process (BEP) is construction of large plants such as those in the Midwestern corn growing states. Transporting biomass is calculated to be a major contributor to the cost of the ethanol produced. This conventional approach also does not utilize the considerable corn, soybean, and other crops grown in the thousands of smaller farms nationwide. Likewise, the current model does not permit relocation of the biomass to ethanol production refineries, as crop yields and planting vary year-to-year especially in non-farm belt states. Another concern with the current model is the construction of permanent facilities with conventional fermentation and distillation equipment that are not readily adaptable to new innovations in the biomass to ethanol process.

### SUMMARY OF THE INVENTION

Fermentation technology has progressed with the generation of yeast or bacteria which ferment hexoses and pentoses. While research continues in these areas, there is the need to focus on ways to enhance the performance of these microbes. One such approach is the removal of ethanol and other inhibitors of fermentation such as acetic acid as they are generated. This invention introduces technology that allows removal of ethanol and organic inhibitors during fermentation resulting in the potential for extended batch fermentation and increased ethanol production. Furthermore, this technology eliminates the need for costly distillation and high water consumption to extract the ethanol. The process also allows the production of biodiesel when eventual replacement is needed. The extracted organic byproducts can be isolated or used as fuel in the biodiesel. The use of solvents including oils to extract ethanol from water is a known technique but has received little attention.

The present invention employs mobile fermentation equipment using technology that allows for extended batch fermentation and efficient ethanol extraction. This invention offers a solution to the transportation challenge by use of ethanol mobile bioreactor units at the site of biomass growth. Individual or small groups of growers can use the mobile units for operation near their fields. Specific housing is not required, only a source of water and electricity. The units can even be set up under tents or other temporary enclosures since the space needed is small and no large or tall equipment is required. The feasibility of small-scale fuel ethanol reactors for use on farms using various feedstocks including cellulosic residues has been explored and found feasible. However, the concept of a portable small-scale refinery for converting trash into electricity has been realized. With support from the U.S. Army, researchers have produced a prototype portable bioreactor which converts trash including food waste into fuel including ethanol to run a generator to produce electricity for use on the battlefield.

The mobile units of the present invention will be composed of simple mobile equipment which has three core components: a biomass pretreatment tank(s); a fermentation tank; and an ethanol extractor. Pretreatment may be done in standard steel drums fitted for heating and stirring. Corn stover or other biomass is shredded and mixed with hydrolyzing chemicals such as NaOH in water and heated to near 100 C with stirring. After lignin separation has progressed, hot vegetable oil or other ethanol-miscible liquid from the ethanol extractor is added to further separate the dissolving lignin. The alkali/oil treatment will take several hours. Mixing is stopped and solids allowed to settle. The lignin rich oil and water supernatant is removed and stored for eventual separation of the lignin. In a preferred embodiment, the pH and temperature of the wet solids is adjusted to allow activity of cellulases added for enzymatic hydrolysis over a period of several hours. Hop acids or other antimicrobial agents are used to control bacterial contamination. After enzymatic hydrolysis, the resulting slurry is pumped directly into the fermentation tank.

The fermentation tank may be similar to an above-ground swimming pool and can be as large as 5000 gal., more or less depending on the biomass capacity of each site. Yeast is the microbe of choice for fermentation, not in small part due to the potential to have greater control of bacterial contamination, a major problem in repeated fermentations. Antibiotics like tetracycline are inexpensive and effective in preventing contaminating bacterial growth. However, concern over the development of bacterial resistance makes alternative prevention and treatment desirable. Again, hop acids have been found effective and are environmentally safe. Yeast are incubated in an enriched prefermentation medium and then added to the tank. In the technology of the present invention, vegetable oil or other ethanol-miscible liquid is layered onto the fermentation liquid which maintains anaerobic conditions. The oil is pumped from the center of the tank and is circulated to an extractor to remove ethanol which has dissolved in the oil during fermentation. The hot oil is pumped to the pretreatment tank(s) or oil holding tank as use permits. When the extractor is off-line, the oil or other ethanol-miscible liquid is pumped from the center of the fermenter down to the level of the stirring device where it is mixed with the fermentation liquid and ethanol is extracted from the water as the emulsion rises to the surface.

Fermentation using the present technology is enhanced since ethanol is removed as it is made, thus reducing its inhibitory effect on yeast. Moreover, many of the organic chemicals produced during fermentation and pretreatment are removed by oil extraction. These components contribute to the energy content of the biodiesel made from the spent vegetable oil in the present process. The fermentation can be extended over many days in the same tank due to the removal of the inhibitors. As glucose levels drop, pretreated biomass are added to allow continued fermentation. Once fermentation can no longer be sustained, the oil is drained separately from the aqueous fermentation liquid. The liquid can be stored for treatment or regeneration. The fermentation tank is cleaned and remaining solids allowed to air dry separately for potential use as feed or fuel. The potential for environmental concerns is low since there is no discharge of contaminated water or gases.

Vegetable oil or other ethanol-miscible liquid from the fermentation tank is pumped into a metal heating tank containing copper mesh pads. The oil is heated to over 100 C by an electric coil under the tank. As the oil drips down through the mesh, the ethanol is vaporized and enters the condenser. The ethanol vapor is condensed in tubing which is wrapped around a metal hollow core filled with ice water. Both the heater and condenser chambers are fully insulated from each other and from the exterior. The only heat loss is the hot exhaust oil or other ethanol-miscible liquid; however, the hot oil is pumped to the pretreatment tanks and used to preheat the biomass. The oil from the fermentation tank is an emulsion to some extent. Depending on the amount of water dispersed in the oil or other ethanol-miscible liquid, it may be necessary to demulsify prior to the extraction. Simple standing may be sufficient or use of chemicals may be needed. If water entrapment is small, demulsification may not be needed and some water will be vaporized in the extractor along with the ethanol. In such event, molecular sieves are used to absorb the water in the collecting drums following condensation. If present in large quantities, additional water extraction with molecular sieves or distillation may be provided at a central collection/distribution site where the drums are delivered from the farms by rail or truck.

### BRIEF DESCRIPTION OF THE DRAWINGS

FIG. 1 is a flow diagram illustrating the process of the invention;

FIG. 2 is a diagram of the preferred embodiment of the extractor unit shown in FIG. 1; and

FIG. 3 is a diagram of a drum which may be used in the pretreatment unit of FIG. 1.

### DESCRIPTION OF THE PREFERRED EMBODIMENTS

The apparatus of the present invention is preferably mobile and may comprise separate units mounted on trucks or trailers for transportation to collection sites where the stover or other biomass is produced. Such site may be in the fields where the bioproducts are grown or may be at a station in the vicinity of the fields to which the biomass products are transported by conventional farm equipment.

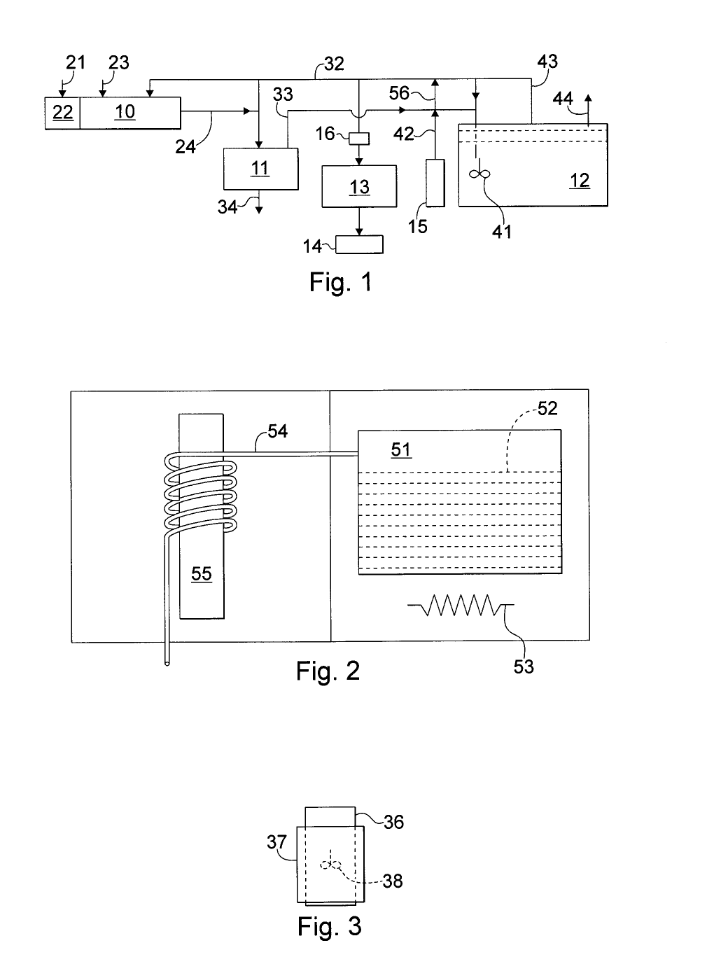

As indicated in FIG. 1, the apparatus is comprised of several units, including a collector 10, pretreatment unit 11, a fermentation reactor 12, an extractor 13, an ethanol receptacle 14 and may include a vegetable oil reservoir 15 and a filter 16.

The collector 10 is adapted to be fed with biomass as indicated by the arrow 21. Preferably, the collector includes a macerator 22 which comminutes the biomass prior to mixing with a hydrolyzer which is introduced into the collector as indicated at 23. The comminuted biomass is thoroughly mixed with the hydrolyzer in the collector and is discharged as indicated at 24 into the pretreatment unit 11. In one embodiment of the invention, the collector 10 may comprise a mobile unit similar to a cement truck having a chipper shredder serving as the macerator. The hydrolyzing agent may be one or more hydrolyzing agents fed into the collector so that it is thoroughly mixed with the biomass as the collector is moved from the field to the pretreatment unit.

In another embodiment of the invention, the collector 10 is combined with the pretreatment unit 11 and may comprise one or more steel drums into which the comminuted biomass is introduced preferably along with hydrolyzing agents which are added in sequence or in combinations with appropriate adjustments in pH, temperature, and mixing conditions to effect conversion of the biomass to fermentable sugars.

In one embodiment of the invention, the pretreatment unit may be a large reservoir in which the biomass may be pretreated. The unit 11 may include stirring means, such as an agitator or mixer, and temperature-regulating means to heat the newly introduced biomass to a suitable temperature to initiate cellulose digestion and lignin separation. When the contents of the reservoir are throughly mixed, the heater or temperature regulator is deactivated or removed and allows the temperature to drop to a temperature suitable for activity of cellulase(s) which are added, and the mixing continues. When thoroughly mixed, the pretreated material is allowed to cool to about 30° C. so that it may be pumped from the fermentation reactor 12, as indicated at 33. Any residue in the pretreatment unit may be discharged as indicated at 34

As an alternative to the large reservoir described above, and as diagrammed in FIG. 3, the pretreatment unit 11 may comprise one or more drums 36 which are heated with a temperature regulator comprising an external collar 37 or other external or internal heating device and have a mixer 38 to thoroughly mix the biomass before being pumped to the fermentation unit 12. This alternative is suited for mounting on trucks or trailers, which may be moved to individual fields, as each field is harvested.

The fermentation unit 12 will preferably comprise a tank having the capacity to ferment a suitable size of a batch from the output from the pretreatment unit, for example, 5,000 gallons. The tank has a stirrer as indicated at 41, and the output 33 from the pretreatment unit 11 along with yeast or other fermenting agent which has been incubated in an enriched prefermentation medium is introduced into the tank adjacent to the stirrer so as to insure mixture of the newly added material into the existing contents of the tank. As indicated at 42, vegetable oil or other ethanol-miscible liquid from the reservoir is introduced in a sufficient quantity to form a layer of oil or other ethanol-miscible liquid atop the fermentation liquid in the body of the tank. The oil layer keeps the fermentation liquid air free and serves to collect the ethanol which is generated in the fermentation process. The ethanol is miscible in the vegetable oil, so that it may dissolve in the oil and be entrapped in the layer as the oil rises to the top of the fermentation liquid. The oil is extracted from the top of the tank as indicated at 43 and a vent is provided at 44 to transfer carbon dioxide or other gaseous output from the fermentation process to capture or further process the discharged carbon dioxide, ethanol vapor, or other gaseous effluent, as desired.

Fermentation using the technology of the present invention is enhanced since ethanol is removed as it is made, thus reducing its inhibitory effect on yeast. Moreover, many of the organic chemicals produced during fermentation and pretreatment will be removed by oil extraction. These components will contribute to the energy content of biodiesel which can be made from the spent vegetable oil in the process of the present invention. The fermentation can be extended over many days in the same tank due to the removal of the inhibitors. As glucose levels drop, pretreated biomass is added to allow continued fermentation. The duration of the extended batch fermentation needs to be determined but should last several days. Once fermentation can no longer be sustained, the oil may be drained separately from the aqueous fermentation liquid. The waste water can be stored for treatment or regeneration, for example by solar evaporation. The fermentation tank is cleaned and remaining solids are allowed to air dry separately for potential use as feed or fuel.

Yeast is the microbe of choice for fermentation, primarily due to the potential to have greater control of bacterial contamination, a major problem in repeated fermentations. Antibiotics like tetracycline are inexpensive and effective in preventing contaminating bacterial growth. However, concern over the development of bacterial resistance makes alternative prevention and treatment desirable. Hop acids or other environmentally friendly antimicrobials used in the system of the present invention may be effective.

If it is desired to provide a fermentation unit which is adapted for mounting on trucks or trailers, which may be moved to individual fields as each field is harvested, one or more smaller reservoirs or tanks may be used in the fermentation unit 12. In such case, it may be appropriate to pump the output from the fermentation unit through a purification filter 16 as shown in FIG. 1, in order to remove any microbes prior to entering the extractor unit 13.

The extractor unit 13 is shown schematically in FIG. 2. Vegetable oil from the fermentation unit 12 is pumped into a metal stainless steel heating tank 51 containing copper mesh pads 52. The oil is heated to over 100° C. by an electric coil 53 under the tank 51. As the oil drips down through the mesh, the ethanol is vaporized and enters the condenser through tubing 54. The ethanol vapor is condensed in the tubing 54 which is wrapped around a hollow metal core 55 filled with ice water. Both the heater and the condenser are fully insulated from each other and from the exterior. The only heat loss would be the hot oil exhausted from the heater; however, the hot oil is recycled to the oil reservoir 15, and the collector 10, which in one embodiment of the invention may be combined into one pretreatment tank, where it is mixed with the biomass to preheat it, as indicated at 56 in FIG. 1. The heated vegetable oil and the hydrolyzing chemical(s) gently disrupts the structure of the biomass, primarily to disassociate the lignin from the cellulose and hemicellulose.

Alternatively, the extractor unit 13 may comprise an electric or propane gas water heater. The vapor produced from heating enters a reflux condenser and the heating temperature is adjusted to produce an ethanol-water azeotrope. The reflux condenser is a thin walled, heat resistant plastic tube having a diameter of approximately five centimeters and a length of approximately fifty centimeters. The tube is packed loosely with scrub pads made of copper, other metal, or plastic mesh. The top of the tube is fitted with a thermometer probe (an oven cooking thermometer suffices) to monitor the ethanol-water azeotrope boiling temperature. The azeotrope vapor exits the reflux condenser tube into small diameter plastic tubing of around three meters length in which the vapor condenses and the liquid is collected in a container. When the azeotrope is no longer formed, the distillation is terminated. The hot water contents of the tank are drained for use in the pretreatment unit 10 to enhance digestion as biomass is added and the process repeated.

The ethanol collected in the extractor 13 may have entrained water. To remove the water from the ethanol either before sending it to the tanker trucks or before transferring it to permanent storage at 16, it is subjected to dewatering by suitable dewatering apparatus, such as molecular seive.

Various embodiments of the invention gave been specifically described, but the invention is not limited to these embodiments. Changes and modification may be therein and thereto within the scope of the following claims.

1. Mobile apparatus for producing ethanol comprising a pretreatment collector for receiving contents including a biomass comprising cellulosic vegetation, a hydrolyzing agent, said collector having means to effect mixing to combine and comminute said contents to form a comminuted biomass,
said pre-treatment collector having means to heat the contents of the collector, and means to introduce an ethanol-miscible liquid, and digesting agents for forming a pre-treatment product containing simple sugars,
a fermentation reactor for said pre-treatment product including an agitator, means to introduce said pretreatment product including said ethanol miscible liquid into the vicinity of said agitator means, and means to introduce a fermenting agent including at least one microorganism to be mixed with said product by said agitator to thereby produce ethanol which is captured by said ethanol-miscible liquid, said ethanol-miscible liquid separating from said pre-treatment product in said fermentation reactor to form a separate layer of ethanol-miscible liquid in said reactor,
means to separate said ethanol-miscible liquid and the captured ethanol in said layer to form the output from said reactor, and
an extractor comprising a heater and a condenser receiving said output, said heater being adapted to heat the output to the boiling point of said ethanol to form an gaseous distillate and a residual product, and said condenser adapted to receive the gaseous phase of said distillate and liquify it for collection.

2. An apparatus according to claim 1, wherein said fermentation reactor causes the ethanol to mix with any water in the comminuted biomass, said apparatus including means to separate the ethanol from the water.

3. Mobile apparatus according to claim 2, including a reservoir to collect the residual liquid and maintain an elevated temperature in the liquid, a conduit for supplying warm ethanol miscible liquid to said reactor, a second conduit for supplying warm ethanol miscible liquid to said pre-treatment receiver, and means to direct said ethanol miscible liquid from said reservoir into at least one of said conduits.

4. Mobile apparatus according to claim 2, wherein said means to separate the ethanol from the water comprises a molecular sieve.

5. Mobile apparatus according to claim 1, wherein said ethanol-miscible liquid is vegetable oil.

6. A method for producing ethanol comprising supplying a batch including a biomass comprising cellulosic vegetation, hydrolyzing agents and water,
mixing and comminuting said batch to form a biomash,
pre-treating said biomash by introducing a ethanol-miscible liquid, and digesting agents to form a pre-treatment product containing simple sugars,
transferring said product into a fermentation reactor and providing a layer of said ethanol miscible liquid overlying said product in the reactor, mixing a fermenting agent including at least one microorganism into said product and allowing fermentation to thereby produce ethanol which migrates into said layer, and is captured thereby,
outputting said fermented product, and
heating the output to form gaseous ethanol distillate and a residual product, and cooling said gaseous distillate and liquifying it for collection.

7. A method for producing ethanol according to claim 6, including the step of controlling the reactor to cause the output from said fermentation reactor to consist of the ethanol-laden ethanol miscible liquid from said layer substantially free of other components.

8. A method for producing ethanol according to claim 6, including a step of collecting the residual product containing ethanol miscible liquid from the heating step, and supplying said ethanol miscible liquid to at least one of said pre-treatment step, and said fermentation step.

9. A method according to claim 6, wherein said ethanol-miscible liquid is vegetable oil.

10. A method according to claim 6 including the step of heating and cooling the comminuted biomass prior to fermentation.

11. A method according to claim 6 including the step of dewatering the ethanol.
